(19) United States
(12) Patent Application Publication
(10) Pub. No.: US 2009/0259176 A1
(43) Pub. Date: Oct. 15, 2009

(54) TRANSDERMAL PATCH SYSTEM

(75) Inventor: Micah B. YAIRI, Palo Alto, CA (US)

Correspondence Address: LEVINE BAGADE HAN LLP, 2400 GENG ROAD, SUITE 120, PALO ALTO, CA, 94303, US

(73) Assignee: Los Gatos Research, Inc., Mountain View, CA (US)

(21) Appl. No.: 12/100,250

(22) Filed: Apr. 9, 2008

(51) Int. Cl. A61F 13/02; A61M 35/00

(52) U.S. Cl. 604/67; 604/305; 604/307; 604/290

(57) ABSTRACT

A transdermal patch system configured as a patch or pump assembly may be placed into contact upon a skin surface to transport drugs or agents transdermally via any number of different mechanisms such as microporous membranes, microneedles, in-dwelling catheters, etc. The assembly may enclose or accommodate a reservoir configured as an elongate microchannel to contain the drug or agent suspended in a fluid vehicle. The reservoir may also be fluidly coupled via microchannels to transport the drugs into or against an underlying skin surface as driven or urged via a pump and controlled by an electronic control circuitry which may be programmed to affect any number of treatment regimens.

### FIELD OF THE INVENTION

The present invention relates to transdermal patch systems. More particularly, the present invention relates to patch or pump systems or apparatus which may be positioned upon a region of a patient's skin surface and their methods of use to efficiently deliver any number of drug therapies in a controlled manner,

### BACKGROUND OF THE INVENTION

Transdermal delivery of drugs generally allows one or more pharmaceutical agents to be introduced into a patient's system at a controlled rate through the skin. Drug delivery may be effected via a patch which contains a drug which is applied to the patient's skin. The drug may penetrate the skin surface by various passive or active mechanisms. Examples of passive mechanisms may include simple diffusion or absorption through the skin, osmosis, etc., and some examples of active mechanisms may include introduction through the skin via mechanical insertion through needles, abrasion, etc., or through electrical methods such as iontophoresis where a suspension of the drug molecules is subjected to an electric field for passage into or through the adjacent skin surface and into the patient's blood stream. Other methods, such as the use of chemical penetration enhancers, heat, or ultrasound waves, are also typically used to increase drug delivery rates through the skin barrier.

Iontophoresis-based patches use an applied electrical current or voltage to drive pharmaceutical formulations through the skin. The electrical current can be programmed to adjust and control the rate of drug delivery into the patient skin. However, with such patches it can be difficult to control the infusion rate and may or may not be effective depending upon the condition or the patient's skin surface. Moreover, typical iontophoresis-based drug delivery is limited to certain classes of ionic molecules which exclude a wide variety of medications, particularly drugs having a relatively large molecule size.

Other patches or drug delivery pumps include infusion pumps which deliver drugs via a needle or catheter inserted through the skin for conditions such as insulin therapy for diabetes treatment. Such patches or pumps deliver drug molecules via a fluid vehicle where the fluid is typically retained within a reservoir which is coupled to a delivery mechanism such as a membrane, needle, or catheter depending upon the delivery mode. However, use of a typical fluid reservoir may be problematic with respect to maintaining the reservoir under pressure to provide adequate flow through the delivery mechanism.

Moreover, fluid reservoirs may be susceptible to the formation of bubbles within the fluid as well as the angle and orientation of the reservoir relative to the patient's body. A depressurized or non-pressurized fluid reservoir may provide only intermittent or inadequate fluid delivery depending upon the orientation of the delivery mechanism and relative fluid levels. Additionally, typical fluid reservoirs may also provide for a number of failure points through which the fluid may leak thus resulting in lowered drug delivery efficiency.

Accordingly, there exists a need for a transdermal drug delivery apparatus or system which provides for consistent drug delivery without the typical problems associated with such systems and which provides additional controllability to tailor a drug therapy regimen to affect any number of treatments.

### SUMMARY OF THE INVENTION

A patch or pump apparatus or system may be placed into contact upon a region of skin to transport drugs or agents transdermally via a number of different mechanisms such as maintaining simple contact of the drugs or agents upon the skin surface for absorption (either with or without chemical penetration enhancers), iontophoresis, needles, in-dwelling catheters, etc. The patch or pump may be removably adhered or placed directly upon the user's skin surface via any number of methods such as being adhered directly to the skin via a temporary adhesive layer or optionally through direct pressure utilizing a strap or band.

Regardless of the mechanism by which the patch or pump is maintained relative to the skin surface, the system may include a number of features which facilitate drug delivery to the patient. For example, the patch or pump assembly may enclose or accommodate a fluid reservoir within a housing to contain the drug or agent suspended in a fluid vehicle. The reservoir may be fluidly coupled via a microchannel lumen through which fluids or medications may be transported to a transdermal drug delivery mechanism in contact with or in proximity to the underlying skin surface. A pump may be used to drive or urge the drugs from the reservoir and through the skin via one of the drug delivery mechanisms, such as through an array of microneedles. The pump may also be used to deliver the drugs from the reservoir for placement directly upon the skin surface where it may be left in place or maintained in contact against the skin surface for absorption, e.g., by maintaining contact against the skin with a microporous membrane. In placing the drugs against the skin surface (rather than introducing or urging the drugs through the layer of skin), a number of chemical enhancing agents may be utilized to facilitate the absorption of the drugs into the skin surface. For instance, agents such as propylene glycol, ethyl alcohol, dimethyl sulfoxide, etc., may be placed upon the skin surface prior to, during, or after placement of the drugs upon the skin or such agents may be combined with the drugs in the fluid vehicle such that they are delivered along with the drugs directly upon the skin surface.

To control the pumping of the drugs as well as different treatment regiments, electronic control circuitry may also be positioned upon the housing and in electrical communication with the pump. A battery (that may be rechargeable or replaceable) may also be positioned along the housing for providing power to the pump, control circuitry, and any other features as necessary.

The electronic control circuitry may provide a variety of functionality and determines when the pump should be active. By controlling when the pump is active or inactive, the electronic circuitry may be used to control when fluid from the reservoir is pumped to the skin. The control circuitry may also include diagnostic algorithms and indicators, such as monitoring battery power, pump operation, circuit integrity, etc. Yet another element that may be included in control circuitry is the inclusion of an on-chip clock that tracks the time and date to facilitate regulation of the fluid delivery schedule controlled by the microprocessor, particularly for chronotherapeutic drug formulations where delivery of medication is on a timed schedule corresponding to the date and/or time of day. Additionally or optionally, a flow rate monitor may also be included in the control circuitry to monitor the amount of fluid which has been delivered upon or through the skin.

Aside from the control circuitry, the microchannels fluidly coupling the various features, such as the reservoir, pump, and microporous membrane may be formed directly within or along the housing and sized to have cross-sectional dimensions ranging anywhere from 1 micron to 5000 microns and lengths varying anywhere from 1 millimeter to 1 meter or longer. Because of their size, the microchannels may facilitate the passage of fluids, such as through capillary action, such that fluid delivery through the channels is consistent regardless of the orientation or angle of the assembly. Moreover, the microchannels may also help to inhibit or prevent the formation of bubbles within the channels such that drug delivery may be metered consistently to the patient.

In yet another variation of the programmable patch or pump assembly, an elongate microchannel may be utilized as the fluid reservoir rather than a single box-like chamber. The microchannel reservoir may be configured into an elongate channel having a cross-sectional dimension ranging anywhere from 1 micron to 1000 microns and a length varying anywhere from 1 millimeter to 1 meter or longer which is looped into an alternating (or “back-and-forth”) pattern which extends over the width and/or length of patch or pump housing. Because of its micrometer scale, the microchannel reservoir reduces the degrees of freedom for liquid movement and constrains the liquid contained within. As a result, altering the orientation of microchannel reservoir in space (three dimensions), such as by rotation or vibration is much less likely to result in the formation of bubbles, gaps, or voids within the contained liquid that may interfere with pumping.

Aside from a single elongate microchannel reservoir which winds in an alternating pattern, the reservoir may be configured into various other patterns as well such as a spiral or any other configuration which allows for fluid storage in or along the housing. For example, the microchannel reservoir may be formed to have one or more separate channels which are aligned parallel to one another. Each of these separate parallel channels may converge into a single microchannel which is fluidly coupled to the pump or other mechanism. The number of channels and the lengths of the individual channels may be uniform or individually varied. Moreover, one or more of the microchannels may contain different drug formulations or varied dosages depending upon the resulting desired dosage and drug combination to be infused into the patient. Yet another variation of a microchannel reservoir may include, e.g., a first microchannel reservoir and a second microchannel reservoir which is separate and distinct from the first microchannel reservoir. Additionally, other variations may include one or more microchannel reservoirs which are aligned along multiple geometric planes within the housing. For instance, a first reservoir may be situated in a first plane along the housing while a second reservoir may be situated in a second plane below or above the first reservoir. In this manner, multiple reservoirs may be “stacked” atop or below one another in several adjacent planes which may be separate from one another or which may be fluidly interconnected between two or more reservoirs between their respective planes. The microchannel reservoirs may each contain the same or different drug formulations or dosages and may each be coupled to one or more valves which may be electronically controlled to meter or control the volume of one or both reservoirs to be pumped.

Use of a microchannel as the drug formulation reservoir may also allow for different types of pump configurations. Rather than using a pump to extract liquid from the microchannel reservoir and pumping it out to ultimately reach a user's skin, an air or gas pump can instead be used to push the liquid down the length of the microchannel reservoir to be ultimately deposited on the user's skin. In a conventional transdermal patch with a single reservoir, use of an air pump is difficult because changing an orientation of the patch or pump may result in pushing air rather than the drug formulation towards the user's skin. This may be avoided because the microchannel reservoir inhibits or prevents bubbles of air from sliding past fluid already contained within the microchannel. Rather, the contained fluid is pushed distally through the microchannel as more air is pumped behind it.

In yet another variation, the microchannel reservoir may be completely removable from the patch or pump. The microchannel reservoir can reside in a removable package or cartridge which may be inserted securely within an interface or receiving channel defined within the housing. Once the reservoir has been depleted, it may be refilled or the cartridge may be removed entirely from the housing and replaced with another cartridge without having to remove the housing from the patient's skin.

In this or any of the variations described herein, the programmable electronic circuitry of the patch or pump assembly may be equipped with a transmitter and/or receiver that allows it to communicate, wirelessly or otherwise, with an external controller such as a computer or hand-held device. The external controller can be used by a physician or the patient to program parameters such as drug delivery time-profiles for a particular patient, customizing the delivery rate profile to a particular patient's needs for a particular medication, etc. Another aspect of the transdermal patch or pump assembly may provide the capability for patient-determined “on-demand” controlled delivery of medication. User-initiated responses may be used as signals to the programmable electronic circuitry to indicate the appropriate dosage profile to be used from that point forward or until a new user-initiated signal is received. These signals may also be used, for example, to initiate an “on-demand” bolus for drug delivery when the user of the patch or pump so desires The amount and rate of drug delivery when the “on-demand” button is pushed may be pre-determined by the circuitry. When a patient desires a small dose of the medication to be administered (such as may the case for an analgesic to relieve pain or a stimulant to help maintain awareness and remain alert), the patient may actuate a control such as pushing a button on the transdermal patch or pump to release a preset bolus of the medication. The control may be part of the electronic control itself.

### BRIEF DESCRIPTION OF THE DRAWINGS

FIGS. 1A and 1B illustrate partial cross-sectional side and top views, respectively, of one variation of a programmable transdermal patch or pump system.

FIGS. 2A and 2B illustrate partial cross-sectional side views of patch or pump system which may be formed of two or more sections which are coupled to one another.

FIGS. 3A and 3B illustrate top and side views of a variation of a cover positionable over the fluid reservoir and having a gas-permeable membrane or layer to facilitate fluid delivery from the reservoir.

FIG. 4A illustrates a detail perspective assembly view of another variation of a drug delivery mechanism utilizing a microneedle array for insertion into the skin surface and an adhesive layer around the array for maintaining consistent contact between the array and skin surface.

FIG. 4B illustrates a partial cross-sectional side view of a transdermal patch or pump system with the delivery mechanism of FIG. 4A.

FIGS. 5A and 5B illustrate partial cross-sectional side and top views, respectively, of another variation of the patch or pump system which utilizes a reservoir comprised of an elongate fluid microchannel.

FIG. 6 illustrates an example of a cross-sectional view showing the relative positioning of the fluid microchannel.

FIG. 7 illustrates another variation of a microchannel reservoir having a parallel channel configuration.

FIG. 8 illustrates a top view of another variation of a patch or pump having at least two separate microchannel reservoirs.

FIG. 9 illustrates a top view of yet another variation showing the infusion of air to push the fluid through the microchannel reservoir.

FIG. 10 illustrates a top assembly view of another variation showing a microchannel reservoir configured as a removable and/or replaceable cartridge from the patch or pump assembly.

FIG. 11 schematically illustrates an example of the patch or pump assembly configured to be controlled wirelessly via a remotely operated control.

FIG. 12 schematically illustrates another example of a patch or pump which utilizes an “on-demand” actuator operable by the patient.

FIG. 13 schematically illustrates another example where the “on-demand” feature is operable by the patient via a remotely operated control.

FIGS. 14A and 14B illustrate top views of variations where the fluid is urged from the reservoir via a linear actuator which may also optionally incorporate a positioning detection system.

### DETAILED DESCRIPTION OF THE INVENTION

In delivering drugs or agents into a patient body over any extended period of time, a patch or pump apparatus or system may be placed into contact upon a region of the user's skin surface and used to transport the drugs or agents transdermally via a number of mechanisms such as maintaining simple contact of the drugs or agents upon the skin surface for absorption (either with or without chemically enhanced absorption), iontophoresis where an applied electrical current or voltage drives the drugs or agents through the skin, membranes, needles, in-dwelling catheters, etc. The patch or pump may be removably adhered or placed via any number of methods directly upon the user's skin surface, e.g., along the arms, legs, hips, abdomen, etc. For example, a portion of the patch or pump or the entire apparatus may be adhered directly to the skin via a temporary adhesive layer or optionally through direct pressure utilizing a strap or band. Alternatively, the patch or pump may be held or adhered to the patient's clothing in proximity to the user's skin in which case a catheter or microneedle array may be used to deliver the drug or agent to the patient through the skin.

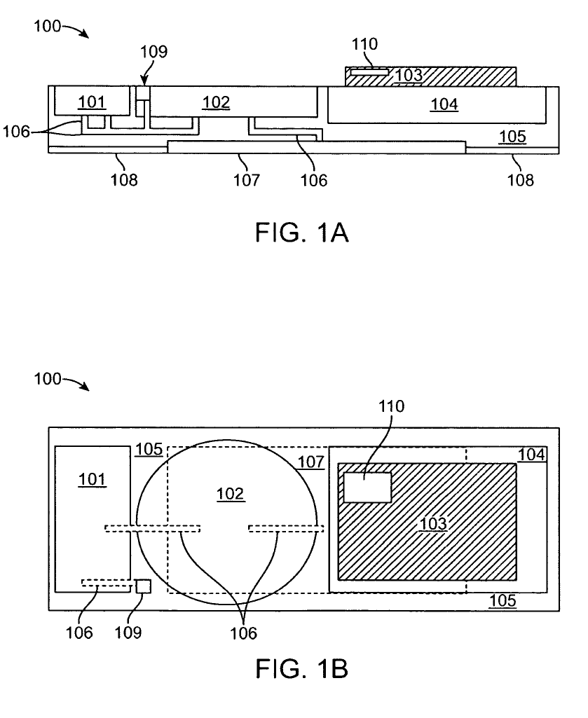

Regardless of the mechanism by which the patch or pump is maintained relative to the skin surface, the system may include a number of features which facilitate drug delivery to the patient. An example is shown in the partial cross-sectional side and top views of FIGS. 1A and 1B, respectively, which illustrates transdermal patch assembly 100 having a housing 105 fabricated from a number of materials including metals or metal alloys such as stainless steel, titanium, etc., or plastic materials polyvinylchloride (PVC), acrylonitrile-butadiene-styrene (ABS), polycarbonate (PC), poly(methyl methacrylate) (PMMA), etc. These materials are merely illustrative of the various types of materials which may be utilized and is not intended to be limiting. Other suitable materials are intended to be included within this disclosure.

For illustrative purposes, housing 105 is shown in a rectangular configuration. In this example, housing 105 may be, e.g., 7 cm in length, 2.5 cm in width, and 1 cm in height. These dimensional values are given merely as examples and housing 105 is not intended to be constrained by dimensional limitations. Accordingly, housing 105 may be dimensioned according to any practicable variation and is intended to be included within this disclosure. Likewise, housing 105 may be configured into various shapes aside from that shown. For instance, housing 105 may alternatively be configured into other shapes, e.g., hemispherical, oblong, etc., so long as housing 105 may be positioned comfortably upon or in proximity to the user's skin surface. In either case, patch assembly 100 may enclose or accommodate fluid reservoir 101 within housing 105 to contain the drug or agent suspended in a fluid vehicle. Reservoir 101 may be fluidly coupled via a lumen such as microchannel 106 to reservoir input port 109 through which fluids or medications may be introduced to fill or refill reservoir 101. Pump 102, such as a peristaltic-type pump, may be fluidly coupled also via a lumen such as microchannel 106 to fluid reservoir 101 to transport or urge the drug or agent through microchannel 106 to a transdermal drug delivery mechanism in contact with or proximity to the underlying skin surface.

Pump 102 is illustrated in this example as fluidly coupled to a microporous membrane 107 which may be placed into contact directly against a skin surface to maintain contact between the drugs pumped from reservoir 101 and the skin. The drugs may simply be left in place or maintained in contact for absorption, e.g., by maintaining contact against the skin with microporous membrane 107. In placing the drugs against the skin surface (rather than introducing or urging the drugs through the layer of skin), a number of chemical enhancing agents may be utilized to facilitate the absorption of the drugs into the skin surface. For instance, agents such as propylene glycol, ethyl alcohol, dimethyl sulfoxide, etc., may be placed upon the skin surface prior to, during, or after placement of the drugs upon the skin or such agents may be combined with the drugs in the fluid vehicle such that they are delivered along with the drugs directly upon the skin surface. Microporous membranes 107 may include membranes having pores in the micrometer range and having a height which extends beyond housing 105 for contacting the underlying skin surface while being attached to housing 105.

In yet other examples, microporous membrane 107 may be omitted entirely from housing 105 and the drugs pumped from reservoir 101 may be deposited directly upon the skin surface. In this case, the drugs may be simply left upon the skin to be absorbed eventually or with any of the chemical enhancing agents mentioned above to facilitate the absorption into the skin.

As mentioned above, assembly 100 may be maintained against the user's skin surface via a strap or band or via an adhesive layer 108 placed directly along housing 105 for contact against the skin surface. Adhesive layer 108 may be comprised of a layer of double-sided adhesive which is attached to the undersurface of housing 105. To control the pumping of the drugs as well as different treatment regiments, an electronic control circuitry 103 may also be positioned upon housing 105 and in electrical communication with pump 102. A battery 104 (rechargeable or replaceable) may also be positioned along housing 105 for providing power to pump 102, control circuitry 103, and any other features as necessary.

Electronic control circuitry 103 may be directly accessible to the user or certain functions may be accessible to the user. Alternatively, circuitry 103 may be completely disabled to the user such that only a physician or other appropriate medical personnel may access the functions or programming features of circuitry 103. In either case, control circuitry 103 may include a processor and/or memory components to control various features of assembly 100. The electronic control circuitry 103 may comprise a printed circuit board with a programmable microcontroller, voltage converter, diagnostic systems, indicators (e.g., light emitting diodes), control switches, actuators, etc. The design and fabrication used to create the electronic control circuitry 103 is standard to those skilled in the art of circuit design.

The electronic control circuitry 103 provides a variety of functionality and determines when the pump 102 should be active. By controlling when pump 102 is active or inactive, electronic circuitry 103 may be used to control when fluid from reservoir 101 is pumped to the skin. Varying the pumping rate adjusts the effective delivery rate of a drug formulation. For example, control circuitry 103 may be programmed to deliver a particular dosage where drug delivery is to be delivered over a specified period of time which may be either preset or determined by the prescribing physician or other appropriate medical personnel. The dosage function may be selected from several preset dosage profiles programmed in a microcontroller within control circuitry 103 or the dosage profile may be customized and entered into control circuitry 103 via a user interface. The control circuitry 103 may also contain input pins to program the microprocessor with customized or tailored drug delivery profiles.

The control circuitry 103 may also include diagnostic algorithms and indicators, such as monitoring battery power, pump operation, circuit integrity, monitoring fluid flow, etc. Yet another element that may be included in control circuitry 103 is the inclusion of an on-chip clock that tracks the time and date to facilitate regulation of the fluid delivery schedule controlled by the microprocessor, particularly for chronotherapeutic drug formulations where delivery of medication is on a timed schedule corresponding to the date and/or time of day. An on-chip clock may also be utilized to regulate drug delivery rates based upon an input of a user's circadian rhythms. If multiple patches or pumps are worn sequentially, e.g., a new patch each day, the clock may be used to provide a changing drug delivery profile that depends on the particular date or the particular number of days that has passed since the first patch or pump was used or according to a customized dosage delivery profile.

A related implementation of control circuitry 103 may include a user-activated interface control such as a button which may be programmed to start or activate a treatment regiment for a specified period of time, e.g., a sleep button. These user-initiated responses may be used as signals to the microprocessor to indicate the appropriate dosage profile to be used from that point forward or until a new user-initiated signal is received. These signals may also be used, for example, to indicate the start or ending of a part of the user's circadian rhythm cycle. Alternatively, such a control may be used to initiate an “on-demand” bolus for drug delivery when the user of the patch or pump so desires. The amount and rate of drug delivery when the “on-demand” button is pushed may be pre-determined by the microcontroller. The fluid amount delivered may be programmed to provide a gradually reduced amount with subsequent “on-demand” requests as well have a minimum time between “on-demand” responses. These examples are intended to be illustrative of some of the possible programmable treatment regimens and are not intended to be limiting.

In addition to controlling the various features described above, assembly 100 may further incorporate an optional radio-frequency identification (RFID) antenna and chip assembly 110 either integrated with control circuitry 103 or separately within housing 105 to allow for further wireless control and monitoring of various parameters. For example, information such as the date, time, and dosage of administered medications, drug formulations, or other prescription-related information, etc., may be stored on RFID assembly 110 for wireless transmission to and/or access by the user or physician. With RFID assembly 110 in electrical communication with control circuitry 103, the user and/or physician may also actively and wirelessly alter parameters such as the patient's dosage depending upon the patient conditions. Examples of such RFID chip assemblies 110 may be commercially available such as the ISIS PATCH as manufactured by Isis Biopolymer, Inc. (Warwick, R.I.).

Aside from control circuitry 103, the microchannels 106 fluidly coupling the various features, such as reservoir 101, pump 102, and microporous membrane 107 may be formed directly within or along housing 105 and sized to have cross-sectional dimensions ranging anywhere from 1 micron to 1000 microns and lengths varying anywhere from 1 millimeter to 1 meter or longer. Microchannels 106 may be configured to have a cross-sectional height and width or it may be configured into circular shapes as well. In other alternatives, separate tubes or lumens having such dimensions may be utilized to transport the fluids accordingly. The use of microchannels 106 to transport fluids within assembly 100 may provide for efficient fluid transport. Because of their size, microchannels 106 may facilitate the passage of fluids, such as through capillary action, such that fluid delivery through the channels is consistent regardless of the orientation or angle of assembly 100. Moreover, microchannels 106 may also help to inhibit or prevent the formation of bubbles within the channels such that drug delivery may be metered consistently to the patient.

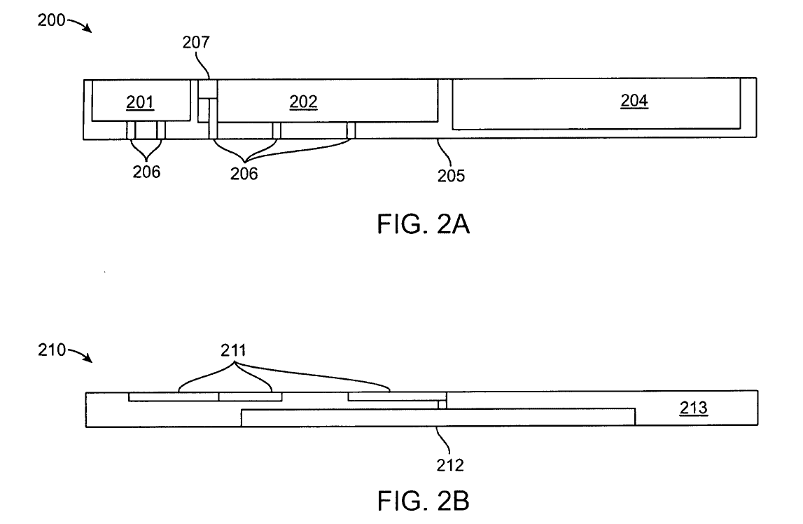

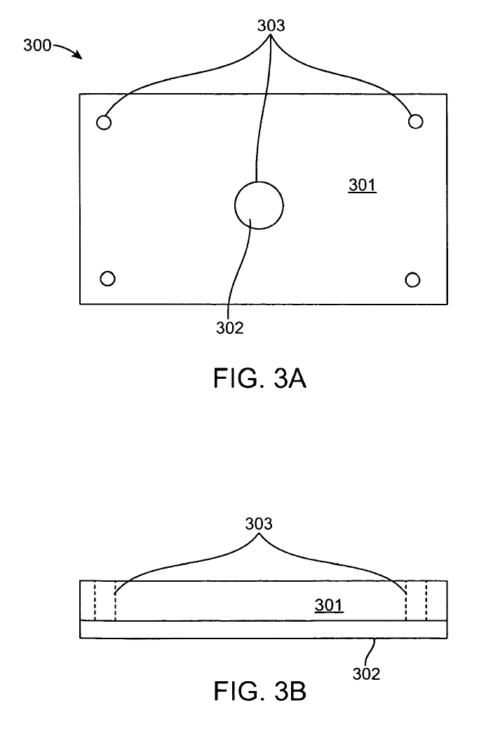

In assembling the patch or pump, housing 105 may be fabricated into a continuous and integral unit by mechanically forming the housing, e.g., by drilling, machining, injection molding, etc., or by other methods such as stereolithography. In other methods, the housing may be fabricated from two or more separate sections which may be attached or coupled to one another to form a single unit. For example, FIGS. 2A and 2B illustrate partial cross-sectional side views of first housing assembly 200 and second housing assembly 210, respectively, which may be joined together to form a single housing assembly for the patch or pump. In one example, first and second housing assemblies 200, 210 may be both fabricated from any of the suitable materials above such that they are made from the same material or from different materials.

In either case, first housing assembly 200 may have materials removed or cut from the starting block or it may be formed to create appropriately sized channels such as channels 201, 202, 204, 207 to form locations for the reservoir 101, pump 102, battery 104, and reservoir input port 109, respectively. To form microchannels 206 extending from their respective channels 201, 202 of the resulting housing 205, stereolithography may be utilized to maintain smooth microchannel surfaces. Other methods for forming the housing assemblies and/or channels may also include a number of other manufacturing processes, such as injection molding, stamping, micromachining, etc. Similarly, second housing assembly 210 may be processed to create microchannels 211 as well as channel 212 for microporous membrane 107. The resulting housing 213 may be joined, coupled, or otherwise attached (e.g., thermally annealed, mechanically coupled, adhered via adhesives, etc.) to housing 205 to create a single patch assembly. The microchannels 206, 211 may also be optionally coated or chemically altered prior to being sealed to make the channel walls more hydrophilic or hydrophobic as desired to make them react in a particular manner with a particular fluid formulation. Alternatively, various other coatings may be applied to enhance other characteristics of microchannels 206, 211.

With the two sections of housing, 205, 213 attached to one another, the remaining components may be included. For instance, pump 102 may be aligned within channel 202 so that its inlet port connects to the microchannel 206 leading from the reservoir 101 and its outlet port connects to the microchannel 206 leading to the microporous membrane 107 within channel 212. Depending upon the particular type of pump utilized, gaskets such as rubber O-rings may be used to seal openings leading to or from pump 102. Alternatively, if the pump's inlet and outlet ports extend perpendicularly relative to pump 102, the sidewalls of the ends of the microchannels 206 and the ports of pump 102 may form a natural seal.

During use of the patch or pump assembly when fluid is pumped, fluid reservoir 101 may be sealed such that it is fluid tight yet gas-permeable to allow for the introduction of a gas such as air to prevent or inhibit the formation of a vacuum within reservoir 101. One variation may include securely placing or sealing a lid assembly 300 upon fluid reservoir 101 where lid 301, which may be made from any of the suitable materials described above, may having one or more openings 303 drilled or otherwise formed through lid 301. A gas-permeable membrane 302, e.g., gas-permeable TEFLON® (E. I. DuPont De Nemours, Wilmington, Del.) may be placed upon or secured to lid 301 such that the one or more openings 303 are covered by membrane 302. Thus, assembly 300 may be secured over fluid reservoir 101 to maintain a fluid-tight seal yet allow for gas to be infused into reservoir 101 through openings 303 via membrane 302 to prevent the formation of a vacuum within the reservoir 101.

Alternatively, fluid reservoir 101 may be formed by a reconfigurable membrane which collapses upon itself as fluid is drained from the reservoir 101. In yet another variation, one of the walls of fluid reservoir 101 may be movable such that as the fluid level is decreased within reservoir 101, the wall may be urged, e.g., via a spring, to reduce the volume of reservoir 101.

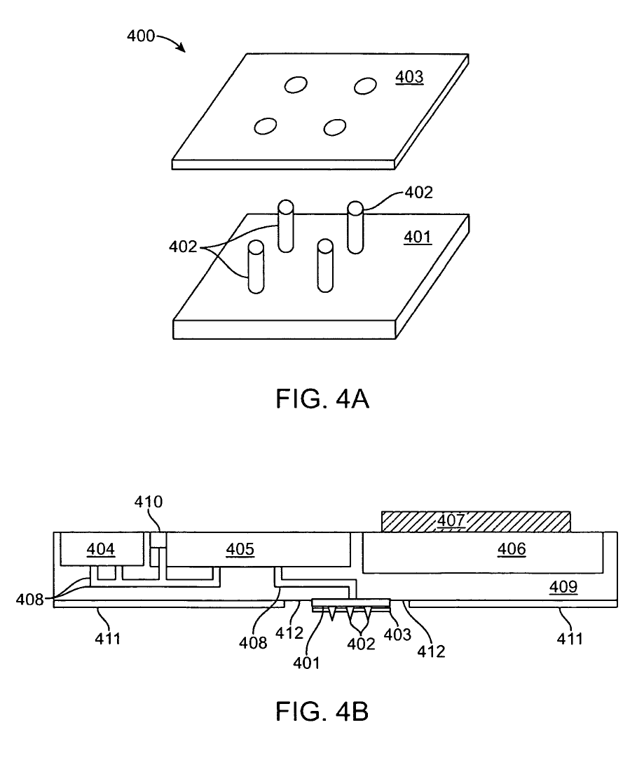

In other variations of the patch or pump assembly, rather than using a microporous membrane 107, an array of microneedles may be used to transport the drug or agent transdermally through the underlying skin surface. FIG. 4A illustrates a perspective assembly view of an example of a microneedle assembly 400 having a microneedle array 401 with one or more microneedles 402 extending therefrom. The one or more microneedle 402 are typically made from silicon and may have an inner and outer diameter in the micrometer range. Because it is generally desirable to maintain insertion of the microneedles 402 within the skin for the duration of time when the patch or pump is positioned upon the skin surface, an adhesive sheet or film 403 may be placed upon the base of array 401 directly around or adjacent to microneedles 402. Corresponding holes or openings may be formed through the adhesive sheet 403 so that it may be placed against the base of the microneedle array 401 without interfering with microneedles 402 and also without resulting in excess material building up at the base of the microneedles 402. Microneedle assembly 400 may be secured upon the patch or pump assembly, as shown in the partial cross-sectional side view of FIG. 4B, such that microneedles 402 extend into and/or through the skin surface when the assembly is secured upon the skin. Moreover, assembly 400 may be removably attached to the patch or pump assembly to allow for replacement of the assembly 400.

An example of a patch or pump assembly is illustrated having fluid reservoir 404 fluidly coupled to pump 405 and to microneedle assembly 400 via microchannels 408 through housing 409. Reservoir port 410 and battery 406 as well as control electronics 407 are also illustrated. Microchannels 408 may deliver the fluid suspension through the microneedles 402 and through the patient skin surface. The patch or pump assembly may be simply secured to the patient body via a support band or strap, as described above, and/or via an adhesive layer 411 placed along the portion of housing 409 in contact with the skin.

Adhesive sheet 403 may adhere directly to the portion of the skin through which microneedles 402 are pierced such that the region of skin immediately beneath and/or adjacent to microneedles 402 are stabilized relative to microneedles 402 to inhibit any motion which may occur if the patch or pump is moved relative to the skin surface or from any muscle movement. This localized isolation of the skin surface relative to the inserted microneedles 402 may inhibit or prevent tearing of skin or damage to the microneedles 402 themselves. Moreover, adhesive sheet 403 may localize the securement of the skin surface to stabilize microneedles 402 while allowing for a greater degree of flexibility of the remaining skin relative to housing 409. This in turn provides for greater patient movement comfort and greater comfort. Additionally, if adhesive layer 411 is utilized along the contacting surface of housing 409 along with adhesive sheet 403, a gap or space 412 may separate the two layers effectively isolating adhesive sheet 403, as shown, thus leaving the skin between adhesive sheet 403 and adhesive layer 411 relatively free to move. While adhesive sheet 403 secures the skin immediately beneath and/or adjacent to microneedles 402, gap or space 412 may provide for added flexibility of housing 409.

Other methods for applying an adhesive layer may include temporarily covering microneedles 402 and/or subsequently spraying on a biocompatible adhesive to microneedle array 401 or wicking a liquid adhesive along the base of array 401 in-between microneedles 402. In addition to adhesive sheet 403, an additional adhesive layer 411 may also be placed along the surface of housing 409 for secure placement against the skin surface. The additional adhesive layer 411 may be integrated along microneedle assembly 400 or it may be separate from the microneedle array 401 such that assembly 400 is removably replaceable.

In another variation of the patch or pump assembly, rather than utilizing a single integrated assembly, the housing may be separated into two or more discrete sections which are connect by wire or capillary tube where appropriate. For example, the reservoir 404, pump 405, battery 406, and electronic control 407 might reside in a first section while microporous membrane or microneedle assembly might resides in a separate second section. This configuration may allow for a relatively lighter and more flexible patch to be used where the drug formulation directly interacts with the skin.

Yet another variation may omit a microporous membrane or microneedle assembly entirely. Typical passive drug delivery patches utilize membranes to prevent the contents of the patch from becoming absorbed at an uncontrolled rate through the skin surface without any regulation at all. It also often serves to contain the liquid in the patch itself to form part of the effective barrier of the drug formulation reservoir. However, in the present patch or pump assembly, the rate of delivery may be strictly limited by the pump 405 even when the microporous membrane or microneedle assembly is omitted entirely. As a result, a programmable transdermal patch may be designed in which the output of the pump 405 leads directly to the skin without any intervening delivery interface. The liquid is spread across the area of the skin within the surrounding boundary of the adhesive layer 108 by, e.g., gravity, diffusion, surface tension, and/or rubbing against the base of the programmable transdermal patch or pump.

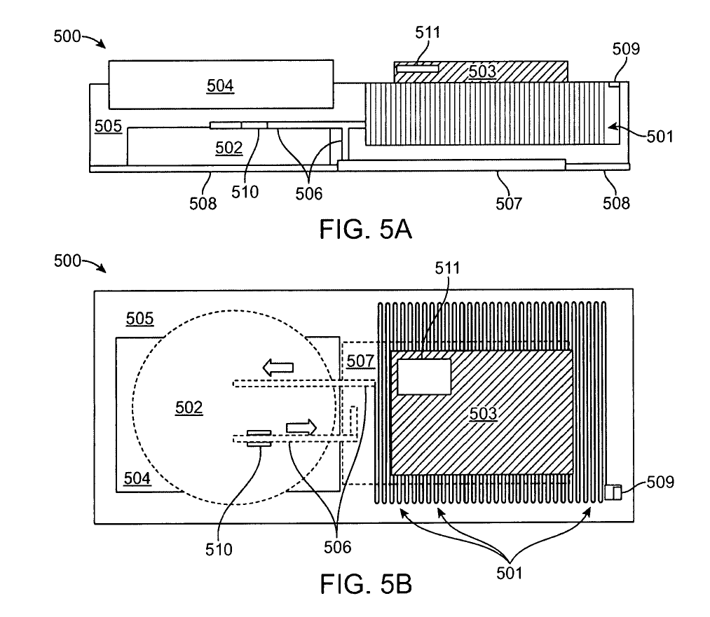

In yet another variation of the programmable patch or pump assembly, FIGS. 5A and 5B illustrate partial cross-sectional side and top views, respectively, of a variation 500 which utilizes a fluid reservoir which is configured as an elongate microchannel 501 rather than as a single box-like chamber. Also shown is RFID antenna and chip assembly 511 optionally incorporated into electronic control 503 which may be used to wirelessly monitor and/or control various parameters of the patch or pump assembly, as described above. Microchannel reservoir 501 may be used to not only transport liquid from one location of the patch or pump to another location, but in this variation the microchannel itself may be utilized as a fluid storage location. Microchannel reservoir 501 may be configured into an elongate channel having a cross-sectional dimension ranging anywhere from 1 micron to 5000 microns and a length varying anywhere from 1 millimeter to 1 meter or longer which is looped into an alternating (or “back-and-forth”) pattern which extends over the width and/or length of patch or pump housing 505. The elongate microchannel 501 may be accordingly configured to retain a fluid volume ranging anywhere from 1 nanoliter to 5 milliliters.

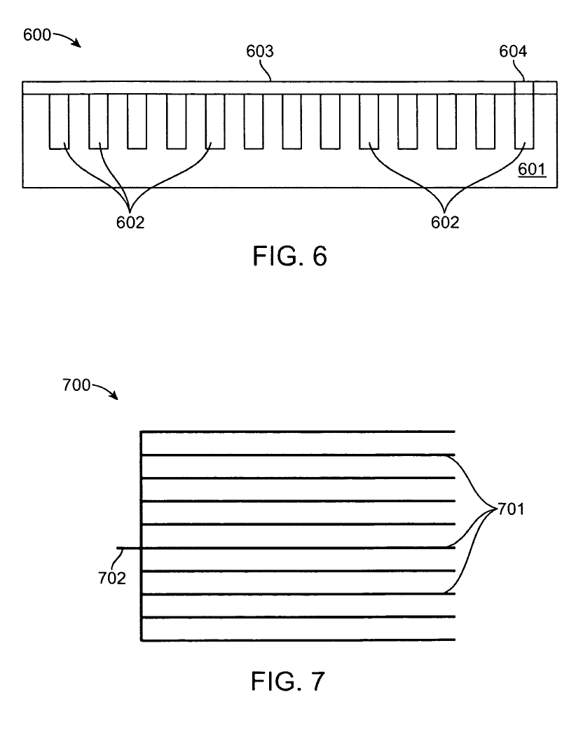

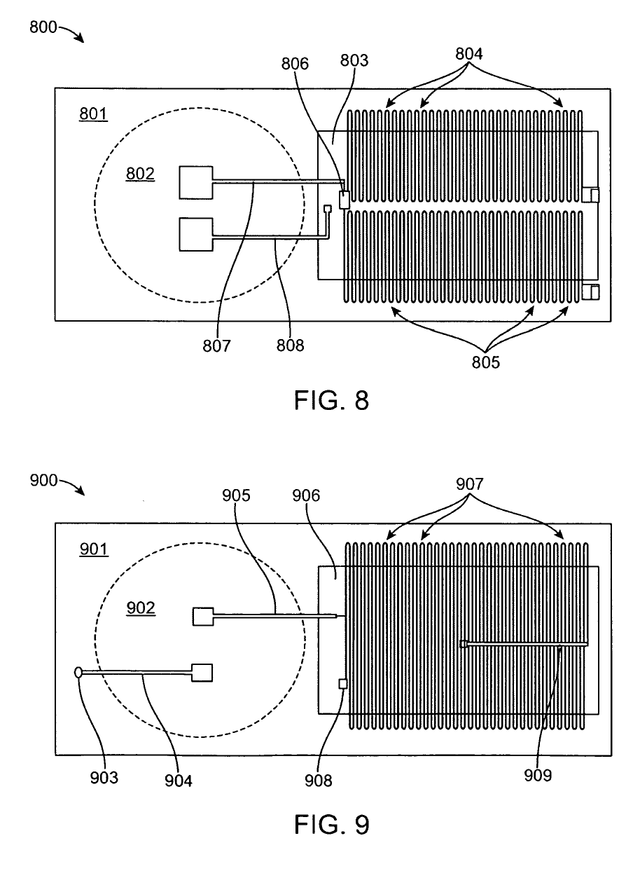

FIG. 6 illustrates an example of a cross-sectional view of housing 601 to show how microchannel reservoir 602 may be aligned relative to one another in its back-and-forth pattern. With reservoir channels formed within housing 601, reservoir 602 may be covered via barrier or lid 603 which may also define a fluid tight and gas-permeable membrane 604 which allows for liquid to be injected into reservoir 602. Turning back to FIGS. 5A and 5B, a proximal end of microchannel reservoir 501 may be covered by the gas-permeable septum or barrier 509 through which fluid may be introduced to fill reservoir 501 and which may also allow for the infusion of a gas, such as air, into the reservoir 501 as the fluid level is depleted through its distal end which may be coupled to pump 502.

Because of its micrometer scale, microchannel reservoir 501 reduces the degrees of freedom for liquid movement and constrains the liquid contained within. As a result, altering the orientation of microchannel reservoir 501 in space (three dimensions), such as by rotation or vibration is much less likely to result in the formation of bubbles, gaps, or voids within the contained liquid that may interfere with pumping. Moreover, the walls of microchannel reservoir 501 may also be coated or modified to increase or decrease their hydrophobic or hydrophilic properties. Also illustrated are pump 502 and microporous material 507 (or any other fluid delivery mechanism) in fluid communication with microchannel reservoir 501 also via microchannels 506. Electronic control 503 and battery 504 as well as adhesive layer 508 are also shown.

Additionally, a fluid detector 510 may also be optionally included within patch or pump assembly 500 to detect the presence of fluid or air within at least one of the microchannels 506. Alternatively, fluid detector 510 may also be included along a portion of microchannel reservoir 501 to detect whether reservoir 501 is empty. In one example of how fluid detector 510 may operate, detector 510 may comprise two metallic or otherwise conductive surfaces positioned in apposition to one another, such as on opposite sides of the output microchannel 506 that connects pump 502 to microporous membrane 507. In the presence of liquid, the resistivity between the electrodes drops while in the presence of air or a bubble, the resistance rises. This resistivity measurement can be used to ensure that if bubbles, voids, or air is introduced into pump 502 and pushed downstream, the subsequent absence of liquid can be detected and pump 502 may be controlled via electronic control 503 to continue pumping until the proper dosage is achieved or an error signal is illuminated for alerting the user. Moreover, fluid detector 510 may be coupled to electronic control 503 to also monitor and track the volume of fluid which has been delivered from reservoir 501 to microporous material 507 (or other fluid delivery mechanism).

Aside from a single elongate microchannel reservoir which winds in an alternating pattern, the reservoir may be configured into various other patterns as well such as a spiral or any other configuration which allows for fluid storage in or along the housing. FIG. 7 schematically illustrates an example of another pattern where microchannels are formed into a parallel configuration. In this example, microchannel reservoir 700 may be formed to have one or more separate channels 701 which are aligned parallel to one another. Each of these separate parallel channels 701 may converge into a single microchannel 702 which is fluidly coupled to the pump or other mechanism. The number of channels 701 and the lengths of the individual channels may be uniform or individually varied. Moreover, one or more of the microchannels 701 may contain different drug formulations or varied dosages depending upon the resulting desired dosage and drug combination to be infused into the patient.

Yet another variation of a microchannel reservoir is illustrated in the top view of assembly 800 which illustrates multiple reservoirs formed within housing 801, e.g., a first microchannel reservoir 804 and a second microchannel reservoir 805 which is separate and distinct from the first microchannel reservoir 804. Additionally, other variations may include one or more microchannel reservoirs which are aligned along multiple geometric planes within the housing. For instance, a first reservoir may be situated in a first plane along the housing while a second reservoir may be situated in a second plane below or above the first reservoir. In this manner, multiple reservoirs may be “stacked” atop or below one another in several adjacent planes which may be separate from one another or which may be fluidly interconnected between two or more reservoirs between their respective planes. Microchannel reservoirs 804, 805 may each contain the same or different drug formulations or dosages and may each be coupled to one or more valves 806, which may be electronically controlled to meter or control the volume of one or both reservoirs 804, 805 to be pumped via pump 802 to microporous membrane 803 (or other delivery mechanism). The microchannel reservoirs 804, 805 may be fluidly coupled to pump 802 via microchannel 807 and pump 802 may be further fluidly coupled to microporous membrane 803 (or other drug delivery mechanism) via microchannel 808. By alternating the frequency and duration of which reservoir is available to pump 802, as well as the pumping rate and duration, the medication from each of the reservoirs 804, 805 can be independently and nearly-simultaneously controlled and delivered to the patient.

Although a single pump 802 is illustrated for pumping both reservoirs 804, 805, each reservoir may alternatively be coupled to a separate pump for controlling the pumping rate of each reservoir individually. Moreover, two microchannel reservoirs are described for illustrative purposes and additional microchannel reservoirs may also be utilized with a common or separate pumping mechanism in other variations.

Use of a microchannel as the drug formulation reservoir may also allow for different types of pump configurations. Rather than using a pump to extract liquid from the microchannel reservoir and pumping it out to ultimately reach a user's skin, an air pump can instead be used to push the liquid down the length of the microchannel reservoir to be ultimately deposited on the user's skin. In a conventional transdermal patch with a single reservoir, use of an air pump is difficult because changing an orientation of the patch or pump may result in pushing air rather than the drug formulation towards the user's skin. This may be avoided because the microchannel reservoir inhibits or prevents bubbles of air from sliding past fluid already contained within the microchannel. Rather, the contained fluid is pushed distally through the microchannel as more air is pumped behind it.

FIG. 9 illustrates an example of this in the top view of housing 901 which contains pump 902 fluidly coupled to microchannel reservoir 907 via microchannel 905 and to microporous membrane 906 via microchannel 909. Microchannel reservoir 907 may be filled and re-filled via introduction of the drug formulation through septum 908 through which new medication can fill the reservoir 907 via an appropriately-sized needle. As air is taken into pump 902 through intake inlet 903 via microchannel 904, air is pushed through microchannel 905 and into microchannel 907. The fluid contained within is pushed by the air to force the fluid through microchannel 909 and to the microporous membrane 906 (or other drug delivery mechanism).

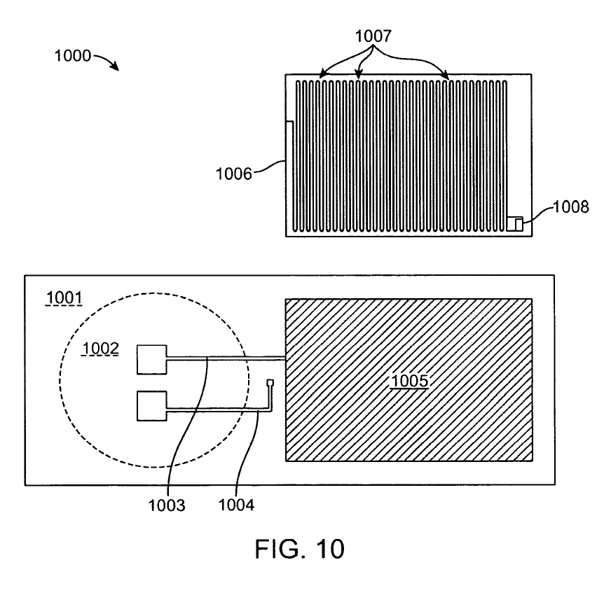

In yet another variation, FIG. 10 illustrates another example where the microchannel reservoir may be completely removable from the patch or pump. As shown, microchannel reservoir 1007 can reside in a removable package or cartridge 1006 which may be inserted securely within an interface or receiving channel 1005 defined within housing 1001 of assembly 1000. Microchannel 1007 may be filled or refilled through septum 1008, which may be gas-permeable as well. Cartridge 1006 may be removably seated within receiving channel 1005 to place microchannel reservoir 1007 in fluid communication with microchannel 1003. Pump 1002 may then pump the fluid through microchannel 1004 to the underlying drug delivery mechanism. Once reservoir 1007 has been depleted, it may be refilled or cartridge 1006 may be removed entirely from housing 1001 and replaced with another cartridge without having to remove housing 1001 from the patient's skin. Moreover, cartridge 1006 may be removed to replace it within housing 1001 to infuse another drug formulation to the patient.

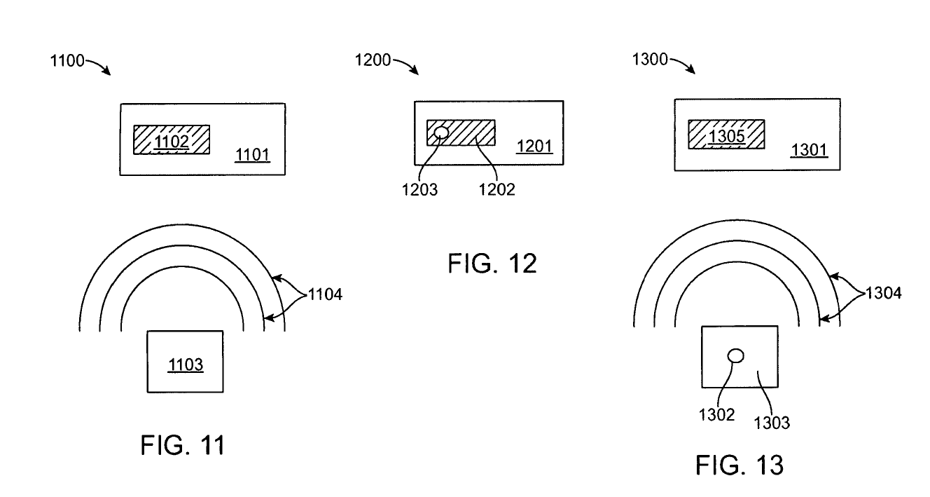

In this or any of the variations described herein, the programmable electronic circuitry 1102 of patch or pump assembly 1101 may be equipped with a transmitter and/or receiver that allows it to communicate, wirelessly 1104 or otherwise, with an external controller 1103 such as a computer or hand-held device, as shown in the schematic illustration of system 1100 in FIG. 11. The external controller 1103 can be used by a physician or the patient to program parameters such as drug delivery time-profiles for a particular patient, customizing the delivery rate profile to a particular patient's needs for a particular medication, etc.

Another aspect of the transdermal patch or pump assembly 1200 may provide the capability for patient-determined “on-demand” controlled delivery of medication, as illustrated schematically in FIG. 12. As previously mentioned above, user-initiated responses may be used as signals to the programmable electronic circuitry 1202 to indicate the appropriate dosage profile to be used from that point forward or until a new user-initiated signal is received. These signals may also be used, for example, to initiate an “on-demand” bolus for drug delivery when the user of the patch or pump so desires. The amount and rate of drug delivery when the “on-demand” button is pushed may be pre-determined by the circuitry 1202. When a patient desires a small dose of the medication to be administered (such as may the case for an analgesic to relieve pain or a stimulant to help maintain awareness and remain alert), the patient may actuate a control such as pushing a button 1203 on the transdermal patch or pump 1201 to release a preset bolus of the medication. The control 1203 may be part of the electronic control itself.

Alternatively, and as illustrated schematically in the system 1300 in FIG. 13, the “on-demand” control 1302 may be part of a remote controller 1303 that may communicate wirelessly 1304 or otherwise with the electronic control circuitry 1305 on the transdermal patch or pump 1301. As above, the amount of medication released when actuated may be preset. For example, it may release the same amount of medication each time or it may release a steadily diminishing amount of medication for consecutive “on-demand” requests. A minimum amount of time may be set between such requests or a maximum number allowed per day, per week, or other specified time allotment. The time between when the “on demand” control is actuated and the actual release of the medication may also be controlled from providing immediate release to a steadily increasing delay.

Aside from the various types of pumps described above for transferring the fluids and drug formulations into or out of a reservoir, linear actuators may be used instead. Generally, such a linear actuator may act as a piston to push or urge the fluids out of the reservoir such that as the piston head is advanced distally as driven by an actuator, voids, vacuum, or air are unable to be mixed with the remaining fluid in the reservoir. Thus, the formation of voids or bubbles may be avoided in the remaining liquid as the reservoir is emptied since the reduction in the volume of the reservoir results in the same volume of liquid (drug formulation) exiting from the reservoir and being deposited onto the skin through any of the methods and devices described herein.

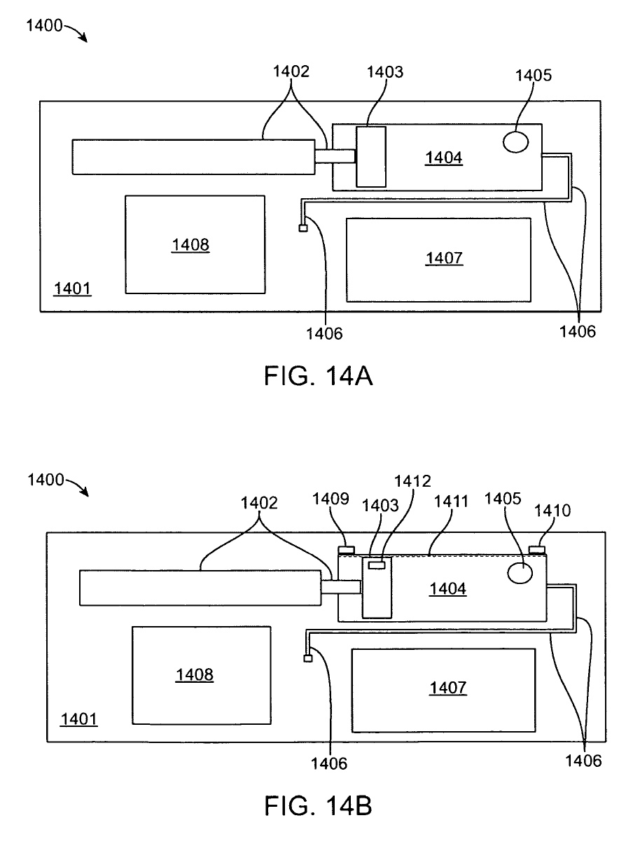

FIG. 14A illustrates a top view of an example of assembly 1400 with housing 1401 containing an electronically-controlled linear actuator 1402 coupled to piston head 1403 and forming one of the walls of reservoir 1404, which may be shaped in various configurations, such as a simple box-shape. Linear actuator 1402 may be powered by battery 1407 and controlled to move via electronic control circuitry 1408. Once actuated by electronic control circuitry 1408, linear actuator 1402 may force piston head 1403 to move distally in a controlled manner while maintaining a seal against the walls of reservoir 1404 such that the fluid contained within the reservoir 1404 is urged through one or more fluidic microchannels 1406 and subsequently into contact against or within the underlying skin surface below, as described above. The reservoir walls and piston head 1403 may form a movable seal via direct contact or via non-contacting surfaces which are in close proximity to one another and which are optionally coated with hydrophobic materials or coatings which inhibit or prevent liquids from seeping between. Additionally, reservoir 1404 may comprise a fluid-tight septum or define an opening 1405 through which reservoir 1404 may be filled.

Linear actuator 1402 may be comprised of any number of actuators, such as a mechanical screw-drive, electromagnetically driven actuation, etc. Another example of a linear actuator which may be utilized with the assemblies described herein are actuators which incorporate a threaded shaft assembly and a threaded nut or carriage which may be subjected to vibrations, e.g., ultrasonic vibrations, which thereby cause the shaft to rotate and/or translate axially. One or more transducers (e.g., piezoelectric, electrostrictive, electrostatic, electromagnetic, etc.) within actuator 1402 may be vibrationally coupled to the nut and/or shaft to force the nut and/or shaft to vibrate at their first mode resonant frequencies. The resulting bending moments may in turn cause the shaft and/or nut to rotate to thereby translate the shaft in a first or second direction. This linear movement may be captured to urge piston head 1403 distally (or proximally) to force the liquid from reservoir 1404. Examples of such vibrationally-driven linear actuators 1402 are described in further detail in U.S. Pat. Nos. 6,940,209; 7,170,214; and 7,309,943, each of which are incorporated herein by reference its entirety.

To accurately determine the amount of fluid delivered at any given time, positioning sensor assembly 1400 may optionally include a system for determining the relative position of piston head 1403 with respect to the reservoir. Given the relative position of piston head 1403 prior to, during, and/or after actuation, the differential volume of fluid delivered from reservoir 1404 may be accurately calculated and/or metered in real-time. Any number of mechanisms may be implemented to determine positioning. One example is illustrated in the top view of FIG. 14B, which shows assembly 1400 with a system optionally incorporated into housing 1401. In this particular variation, one or more electronic sensors 1409 and 1410 may be placed at various positions along reservoir 1404, such as a proximal and distal position along reservoir 1404 relative to the direction of travel of piston head 1403. A capacitive film 1411 may be positioned along reservoir 1404 and in electrical communication with sensors 1409, 1410. As piston head 1403 moves distally through reservoir 1404, it may contact and displace capacitive film 1411 such that any changes in resistance and capacitance between film 1411 and either of the sensors 1409, 1410 may be detected and the relative changes in piston head 1403 position may be determined. Accordingly, the corresponding volume of fluid displaced from reservoir 1404 may be calculated to determine the amount of fluid delivered to or within the skin surface. Alternatively, an emitter or magnet 1412 can be placed within or upon piston head 1403 which may be utilized to determine the position of the piston head 1403 in conjunction with the sensors 1409, 1410.

Although these examples illustrate the use of linear actuators and sensing systems with reservoirs 1404, they may also be incorporated with any of the microchannel reservoirs described above to directly urge the fluids contained therewithin or to indirectly urge the fluids by moving a piston to pump a gas which in turn drives the fluid through the microchannel reservoir, as described above.

The applications of the devices and methods discussed above are not limited to any specific treatments but may include any number of further treatment applications. Moreover, such devices and methods may be applied to various treatment sites upon the body. Modification of the above-described assemblies and methods for carrying out the invention, combinations between different variations as practicable, and variations of aspects of the invention that are obvious to those of skill in the art are intended to be within the scope of the claims.

What is claimed is:

1. A system for transdermal drug delivery, comprising:
a housing configured for placement upon a skin surface,
a drug delivery mechanism within or along the housing positioned to contact the skin surface; and
a reservoir contained within or along the housing and fluidly coupled to the drug delivery mechanism, wherein the reservoir comprises an elongate microchannel extending within or along the housing.

2. The system of claim 1 wherein the housing is comprised of two or more separate sections which are securely attached to one another.

3. The system of claim 1 further comprising a pump in fluid communication with the reservoir and the drug delivery mechanism.

4. The system of claim 3 wherein the pump is further coupled to an opening through which air is introduced.

5. The system of claim 3 further comprising an electronic control circuitry within or along the housing and in electrical communication with the pump.

6. The system of claim 5 wherein the electronic control circuitry is configured to control actuation and/or pumping rates of the pump according to a programmable dosage profile.

7. The system of claim 6 wherein the electronic control circuitry comprises an on-chip clock configured to track a time and/or date of the programmable dosage profile.

8. The system of claim 5 wherein the electronic control circuitry comprises a user-activated control for actuating the pump and/or dosage for a predetermined period of time.

9. The system of claim 5 further comprising a controller separate from the electronic control assembly and in wireless communication therewith.

10. The system of claim 5 wherein the electronic control circuitry further comprises an RFID assembly configured to wirelessly communicate with the pump.

11. The system of claim 3 wherein the pump comprises a linear actuator having a piston head coupled thereto and which is movable within the reservoir.

12. The system of claim 11 further comprising a positioning sensor assembly configured to sense a position of the piston head relative to the reservoir such that a differential volume of the reservoir is determined.

13. The system of claim 12 wherein the positioning sensor assembly comprises a capacitive film contacting the piston head and in electrical communication with one or more sensors positioned along the reservoir.

14. The system of claim 11 further comprising a piezoelectric transducer vibrationally coupled to the piston head such that actuation of the transducer forces the piston head to rotate and/or translate in an axial direction.

15. The system of claim 1 wherein the drug delivery mechanism comprises a microporous membrane having an area configured for contacting the skin surface.

16. The system of claim 1 wherein the drug delivery mechanism comprises a microneedle array projecting from the housing and having a length sized to pierce the skin surface.

17. The system of claim 16 further comprising an adhesive layer beneath and/or adjacent to the microneedle array whereby contact of the layer upon the skin surface immobilizes an underlying portion of the skin surface relative to the microneedle array extending into the portion of the skin surface.

18. The system of claim 17 wherein the adhesive layer is localized upon the skin surface directly about the microneedle array such that a remainder of the skin surface is unrestricted relative to the housing.

19. The system of claim 18 further comprising a second adhesive layer separated from the adhesive layer and which contacts the remainder of the skin surface and immobilizes the remainder relative to the housing.

20. The system of claim 1 further comprising a lid assembly which fluidly seals the reservoir, wherein the lid assembly further comprises a gas-permeable membrane which allows for gas infusion into the reservoir while maintaining a fluid seal.

21. The system of claim 1 wherein the microchannel reservoir has a cross-sectional dimension ranging from 1 micron to 1000 microns.

22. The system of claim 21 wherein the microchannel reservoir has a length ranging from 1 millimeter to 1 meter.

23. The system of claim 1 wherein the microchannel reservoir extends within or along the housing in an alternating back-and-forth pattern over the width and/or length of the housing.

24. The system of claim 1 wherein the microchannel reservoir extends within or along the housing in a spiral pattern.

25. The system of claim 1 wherein the microchannel reservoir has one or more separate channels aligned parallel to one another, wherein each of the one or more separate channels converge into a single microchannel fluidly coupled to the drug delivery mechanism.

26. The system of claim 1 wherein the elongate microchannel is aligned within a first plane along the housing and further comprising at least a second microchannel aligned within a second plane along the housing which is adjacent to the first plane.

27. The system of claim 1 further comprising a second microchannel reservoir separate from the elongate microchannel.

28. The system of claim 27 wherein the elongate microchannel contains a first drug and the second microchannel reservoir contains a second drug different from the first drug.

29. The system of claim 1 wherein the reservoir resides in a package or cartridge removably secured to the housing.

30. A method of delivering one or more drugs transdermally, comprising:
positioning a housing upon or in proximity to a skin surface;
pumping one or more drugs to a drug delivery mechanism placed onto or through a skin surface underlying the housing, and
wherein the one or more drugs are contained within an elongate microchannel reservoir located within or along the housing.

31. The method of claim 30 wherein positioning a housing comprises securing the housing to the skin surface via an adhesive or strap.

32. The method of claim 30 wherein pumping comprises placing a microporous membrane into contact against the skin surface.

33. The method of claim 30 wherein pumping comprises inserting a microneedle array into the skin surface.

34. The method of claim 33 further comprising immobilizing the skin surface relative to the microneedle array extending into the skin surface.

35. The method of claim 34 wherein immobilizing comprises locally immobilizing the skin surface directly about the microneedle array via an adhesive layer such that a remainder of the skin surface is unrestricted relative to the housing.

36. The method of claim 35 comprising further immobilizing the remainder of the skin surface via a second adhesive layer separated from the adhesive layer such that the remainder of the skin surface is immobilized relative to the housing.

37. The method of claim 30 wherein pumping comprises actuating a pump in fluid communication with the microchannel reservoir and the drug delivery mechanism.

38. The method of claim 37 wherein actuating a pump comprises pumping a gas through the microchannel reservoir such that the one or more drugs are pushed towards the drug delivery mechanism.

39. The method of claim 37 further comprising controlling the pump via an electronic control assembly positioned within or along the housing and in electrical communication with the pump.

40. The method of claim 39 wherein controlling comprises controlling actuation and/or pumping rates of the pump according to a programmable dosage profile.

41. The method of claim 39 wherein controlling comprises actuating a user-activated control for actuating the pump and/or dosage for a predetermined period of time.

42. The method of claim 39 wherein controlling comprises remotely controlling the electronic control assembly via a controller separate from the electronic control assembly.

43. The method of claim 39 further comprising wirelessly communicating with an RFID assembly in communication with the electronic control assembly.

44. The method of claim 30 wherein pumping further comprises infusing a gas into a terminal end of the microchannel reservoir while pumping the one or more drugs contained therein.

45. The method of claim 30 wherein pumping comprises urging a linear actuator to drive a piston head in communication with the microchannel reservoir.

46. The method of claim 45 further comprising detecting a relative position of the piston head.

47. The method of claim 45 wherein urging comprises actuating a piezoelectric transducer vibrationally coupled to the piston head such that vibrating the transducer forces the piston head to rotate and/or translate in an axial direction.

48. The method of claim 45 further comprising sensing a position of the piston head relative to the reservoir such that a differential volume of the reservoir is determined.

49. The method of claim 48 wherein sensing comprises electrically detecting a capacitive difference within a capacitive film contacting the piston head and in electrical communication with one or more sensors positioned along the reservoir.

50. The method of claim 30 wherein the microchannel reservoir has a cross-sectional dimension ranging from 1 micron to 1000 microns.

51. The method of claim 30 wherein the microchannel reservoir has a length ranging from 1 millimeter to 1 meter.

52. The method of claim 30 wherein the microchannel reservoir extends within or along the housing in an alternating back-and-forth pattern over the width and/or length of the housing.

53. The method of claim 30 wherein the elongate microchannel is aligned within a first plane along the housing and further comprising at least a second microchannel aligned within a second plane along the housing which is adjacent to the first plane.

54. The method of claim 30 wherein pumping comprises pumping the one or more drugs from one or more separate microchannels aligned parallel to one another.

55. The method of claim 30 wherein pumping comprises pumping an additional drug to the drug delivery mechanism from at least a second elongate microchannel reservoir located within or along the housing separate from the elongate microchannel.

56. The method of claim 30 further comprising removing or replacing a package or cartridge containing the microchannel reservoir from the housing.

57. A system for transdermal drug delivery, comprising:
a housing configured for placement upon a skin surface;
a drug delivery mechanism within or along the housing positioned to contact the skin surface;
a reservoir contained within or along the housing and fluidly coupled to the drug delivery mechanism; and
a linear actuator having a transducer vibrationally coupled to a translatable element positioned within the reservoir, wherein the translatable element is configured to translate within the reservoir in a controlled manner upon vibrational actuation of the transducer.

58. The system of claim 57 further comprising an electronic control circuitry within or along the housing and in electrical communication with the linear actuator.

59. The system of claim 58 wherein the electronic control circuitry is configured to control actuation of the transducer according to a programmable dosage profile.

60. The system of claim 59 wherein the electronic control circuitry comprises an on-chip clock configured to track a time and/or date of the programmable dosage profile.

61. The system of claim 59 wherein the electronic control circuitry comprises a user-activated control for actuating the transducer and/or dosage for a predetermined period of time.

62. The system of claim 59 wherein the electronic control circuitry comprises an RFID assembly in wireless communication with an external controller.

63. The system of claim 59 further comprising a controller separate from the electronic control assembly and in wireless communication therewith.

64. The system of claim 57 wherein the transducer comprises a piezoelectric transducer.

65. The system of claim 57 wherein the translatable element comprises a piston head positioned within the reservoir.

66. The system of claim 57 wherein actuation of the transducer rotates the element such that the element is translated distally in an axial direction within the reservoir such that a volume of the reservoir is decreased.

67. The system of claim 57 further comprising a positioning sensor assembly configured to sense a position of the translatable element relative to the reservoir such that a differential volume of the reservoir is determined.

68. The system of claim 67 wherein the positioning sensor assembly comprises a capacitive film contacting the translatable element and in electrical communication with one or more sensors positioned along the reservoir.
